(19) United States
(12) Patent Application Publication
(10) Pub. No.: US 2024/0325622 A1
(43) Pub. Date: Oct. 3, 2024

(54) REMOTE MONITORING OF FLUID PRESSURE IN BIOLOGICAL TISSUE

(71) Applicant: IPG PHOTONICS CORPORATION, Marlborough, MA (US)

(72) Inventors: ISAAC OSTROVSKY, WELLESLEY, MA (US); GREGORY ALTSHULER, Lincoln, MA (US); DMITRI BOUTOUSSOV, Southborough, MA (US); SERGEI PILIPETSKII, Maynard, MA (US); ILYA YAROSLAVSKY, N. Andover, MA (US); Olivier TRAXER, Paris (FR)

(73) Assignee: IPG PHOTONICS CORPORATION, Marlborough, MA (US)

(21) Appl. No.: 18/579,983

(22) PCT Filed: Jul. 19, 2022
(86) PCT No.: PCT/US2022/037582

§ 371 Date: Jan. 17, 2024

Related U.S. Application Data
(60) Provisional application No. 63/223,251, filed on Jul. 19, 2021.
(60) Provisional application No. 63/228,216, filed on Aug. 2, 2021.

(51) Int. Cl. A61M 3/02; A61M 1/00

(52) U.S. Cl. CPC A61M 3/0202; A61M 1/74; A61M 3/0258; A61M 2205/3337; A61M 2205/3344; A61M 2205/3355; A61M 2210/1082

(57) ABSTRACT

A system for controlling pressure within a kidney that includes an irrigation channel configured with a pressure sensor, a distal end of the irrigation channel in fluid communication with an interior of a kidney, an aspiration channel in fluid communication with a drain reservoir, a distal end of the aspiration channel in fluid communication with the interior of the kidney, and a controller configured to: determine a fluid flow rate of irrigation fluid within the irrigation channel and receive a pressure measurement value from the sensor, calculate a pressure within the interior of the kidney based at least in part on the determined fluid flow rate and the pressure measurement, compare the calculated kidney pressure to a target kidney pressure value, and based on the comparison, control at least one of the fluid flow rates of irrigation fluid in the irrigation channel and the aspiration channel.

### RELATED APPLICATIONS

The present application claims priority to U.S. Provisional Application Ser. No. 63/223,251, titled “REMOTE MONITORING OF FLUID PRESSURE IN BIOLOGICAL TISSUE,” filed on Jul. 19, 2021, and to U.S. Provisional Patent Application No. 63/228,216, titled “REMOTE MONITORING OF FLUID PRESSURE IN BIOLOGICAL TISSUE,” filed on Aug. 2, 2021, the contents of which are herein incorporated by reference in their entirety.

### BACKGROUND

### Technical Field

The technical field relates generally to ureteroscopy, and more specifically a systems and methods for pressure monitoring during these procedures.

### Background Discussion

The pressure inside the kidney increases during ureteroscopy procedures due to the nature of the procedure. Breaking and removing stones from the kidney by using laser energy requires a high fluid flow rate. The fluid is delivered to the interior of the kidney through a working channel of a ureteroscope. The fluid is evacuated from the kidney via a clearance between the exterior surface of the ureteroscope and the patient's anatomy (ureter and urethra inner diameter (ID)), or between the ureteroscope and an interior surface of an access sheath. In both cases the clearance is very small and without an elevated pressure in the kidney, the fluid flow out would be very slow. This requires some pressure to increase the flow, and the higher the pressure, the faster the flow.

The kidney is capable of tolerating a limited pressure. The normal physiological intrarenal pressure is approximately 10 mm Hg (13 cm H₂O). It has been shown that a pressure on the order of 30-40 mm Hg (40-55 cm H₂O) can be safely tolerated, but higher pressures have the potential to harm a patient. A common practice is to use a fluid bag suspended approximately 40 cm above a patient for irrigation. It is important to note that the pressure inside the kidney reaches a maximum level only if the outflow from the kidney is completely stopped. Then the interior kidney pressure becomes equal to the bag pressure (40 cm H₂O), which is considered to be safe. Normally when fluid fills up the kidney, the pressure increases and dilates the anatomy, which opens the clearance between the exterior surface of the scope and the inner surface of the ureter and urethra. This results in outflow, which decreases the pressure inside the kidney. Thus, if fluid comes out of the patient, it is indicative that the pressure inside the kidney is lower than the bag pressure.

It has also been shown that the flow rate through the kidney from the bag at 40 cm above the patient (and with no tool inside of the working channel of the ureteroscope) is typically in a range of 30-40 mL/min. If a laser fiber, guidewire, basket, or any other tool is inside the working channel, then the flow rate decreases to 10-20 mL/min due to the resulting restricted flow of irrigation fluid. The flow rate during laser treatment of kidney stones affects the speed and effectiveness of removal of the stone debris. The higher the flow rate, the shorter the procedure time and the more efficiently debris is removed. It is believed that a flow rate of up to 80-100 mA min is desirable for efficient stone lasing and debris removal. Such flow rates can be reached only by raising the incoming pressure, which can be done if the pressure inside the kidney is controlled in real time. However, while it is possible to place a pressure sensor inside the kidney during the procedure, it is also very difficult and expensive. There is a therefore a need for the ability to monitor and control the pressure inside the kidney remotely, i.e., from outside the patient's body.

### SUMMARY

Aspects and embodiments are directed to a method and system for controlling pressure within a kidney.

In accordance with an exemplary embodiment, there is provided a system for controlling pressure within a kidney that includes an irrigation channel having a proximal end and a distal end and configured with a pressure sensor that is configured to measure pressure within the irrigation channel, the distal end of the irrigation channel in fluid communication with an interior of a kidney so as to deliver irrigation fluid to the interior of the kidney, an aspiration channel having a proximal end and a distal end, the aspiration channel in fluid communication with a drain reservoir, the distal end of the aspiration channel in fluid communication with the interior of the kidney so as to remove irrigation fluid from the interior of the kidney, and a controller in communication with the pressure sensor and configured to: determine a fluid flow rate of irrigation fluid within the irrigation channel, receive a pressure measurement value from the pressure sensor, calculate a pressure within the interior of the kidney based at least in part on the determined fluid flow rate and the pressure measurement, compare the calculated kidney pressure to a target kidney pressure value, and based on the comparison, control at least one of the fluid flow rate of irrigation fluid in the irrigation channel and a fluid flow rate of irrigation fluid in the aspiration channel.

In one example, the irrigation channel is free from internal structures or obstructions. In a further example, the irrigation channel is free from a laser fiber, a guidewire, and a stone retrieval basket.

In one example, the system further includes a flexible sheath having a central passageway for receiving at least a portion of the aspiration and irrigation channels and is configured to be inserted into a ureter, the flexible sheath configured such that irrigation fluid can be drained into the drain reservoir via a drainage flow path between the flexible sheath and the ureter. In a further example, the flexible sheath has a proximal end and a distal end, the distal end of the flexible sheath in fluid communication with the interior of the kidney and the pressure sensor is positioned upstream from the proximal end of the flexible sheath.

In one example, when the calculated kidney pressure is greater than the target kidney pressure value the controller is configured to increase the fluid flow rate of irrigation fluid in the aspiration channel, decrease the fluid flow rate of irrigation fluid in the irrigation channel, or increase the fluid flow rate of irrigation fluid in the aspiration channel and decrease the fluid flow rate of irrigation fluid in the irrigation channel. In a further example, the system further includes an aspiration pump in fluid communication with the aspiration channel and configured to pump irrigation fluid from the distal end toward the proximal end of the aspiration channel, and the controller is configured to control the aspiration pump so as to increase the fluid flow rate of irrigation fluid in the aspiration channel, and one of an irrigation pump or an irrigation fluid flow control valve, each of which is: in fluid communication with a source of the irrigation fluid, in communication with the controller, and is operable to control the fluid flow rate of irrigation fluid in the irrigation channel, and the controller is configured to control at least one of the irrigation pump and the irrigation fluid flow control valve so as to decrease the fluid flow rate of irrigation fluid in the irrigation channel. In a further example, the aspiration pump is configured as a variable speed pump and the controller controls the aspiration pump by powering on or increasing a speed of the variable speed, the irrigation pump is configured as a variable speed pump and the controller controls the irrigation pump by powering off or decreasing a speed of the variable speed, and the controller controls the irrigation fluid flow control valve by restricting or closing the irrigation fluid flow control valve. In a further example, the system further including a drainage channel configured to provide fluid communication between the irrigation channel and the drain reservoir, the drainage channel configured with a safety valve operable to control a flow of irrigation fluid from the irrigation channel to the drain reservoir. In a further example, the controller is configured to control the safety valve by opening the safety valve so as to allow fluid communication between the irrigation channel and the drain reservoir.

In one example, when the calculated kidney pressure is less than the target kidney pressure value, the controller is configured to decrease the fluid flow rate of irrigation fluid in the aspiration channel, increase the fluid flow rate of irrigation fluid in the irrigation channel, or decrease the fluid flow rate of irrigation fluid in the aspiration channel and increase the fluid flow rate of irrigation fluid in the irrigation channel.

In one example, the system further includes a fluid flow rate sensor configured to measure an irrigation fluid flow rate within the irrigation channel, and the controller is further configured to receive a fluid flow rate measurement value from the fluid flow rate sensor and calculate the pressure within the interior of the kidney based at least in part on the fluid flow rate measurement.

In one example, the target kidney pressure value is in a range of 1040 mm Hg inclusive.

In accordance with another exemplary embodiment, there is provided a method for controlling pressure within a kidney that includes directing irrigation fluid through an irrigation channel to an interior of the kidney, removing irrigation fluid from the interior of the kidney and directing the irrigation fluid through an aspiration channel toward a drain reservoir, determining a fluid flow rate of irrigation fluid within the irrigation channel, measuring a pressure within the irrigation channel, calculating a pressure within an interior of the kidney based at least in part on the determined fluid flow rate and the pressure measurement, comparing the calculated kidney pressure to a target kidney pressure value, and based on the comparison, controlling at least one of a fluid flow rate of irrigation fluid in the aspiration channel and a fluid flow rate of irrigation fluid in the irrigation channel.

In one example, when the calculated kidney pressure is greater than the target kidney pressure value the method includes at least one of increasing a fluid flow rate of irrigation fluid in the aspiration channel, and decreasing a fluid flow rate of irrigation fluid in the irrigation channel. In a further example, increasing the fluid flow rate of irrigation fluid in the aspiration channel includes at least one of powering on or increasing a speed of an aspiration pump in fluid communication with the aspiration channel, and decreasing the fluid flow rate of irrigation fluid in the irrigation channel includes at least one of powering off or decreasing a speed of an irrigation pump that is in fluid communication with a source of irrigation fluid, and restricting or closing an irrigation fluid flow control valve that is in fluid communication with the source of irrigation fluid. In another example, the method further includes directing irrigation fluid through a drainage channel that is configured to provide fluid communication between the irrigation channel and the drain reservoir.

In one example, when the calculated kidney pressure is less than the target kidney pressure value, the method includes at least one of decreasing a fluid flow rate of irrigation fluid in the aspiration channel, and increasing a fluid flow rate of irrigation fluid in the irrigation channel. In a further example, decreasing the fluid flow rate of irrigation fluid in the aspiration channel includes powering off or decreasing a speed of an aspiration pump in fluid communication with the aspiration channel, and increasing the fluid flow rate of irrigation fluid in the irrigation channel includes at least one of powering on or increasing a speed of an irrigation pump that is in fluid communication with a source of irrigation fluid, and opening an irrigation fluid flow control valve that is in fluid communication with the source of irrigation fluid.

In one example, the method further includes measuring the fluid flow rate of irrigation fluid within the irrigation channel and calculating the pressure within the interior of the kidney based at least in part on the fluid flow rate measurement.

In one example, the method further includes providing a flexible sheath having a central passageway for receiving at least a portion of the aspiration and irrigation channels, and positioning the flexible sheath within an ureter such that irrigation fluid can be drained into the drain reservoir from a drainage flow path between the flexible sheath and the ureter. In a further example, measuring the pressure within the irrigation channel includes measuring with a pressure sensor in the irrigation channel, and the flexible sheath has a proximal end and a distal end, the distal end of the flexible sheath in fluid communication with the interior of the kidney, and the method further includes providing the irrigation channel, the irrigation channel configured such that the pressure sensor is positioned upstream from the proximal end of the flexible sheath.

In one example, the method further includes providing a ureteroscope that includes the irrigation channel and the aspiration channel, wherein the irrigation channel is configured to be free from internal structures or obstructions.

Still other aspects, embodiments, and advantages of these example aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Embodiments disclosed herein may be combined with other embodiments, and references to “an embodiment,” “an example,” “some embodiments,” “some examples,” “an alternate embodiment,” “various embodiments.” “one embodiment,” “at least one embodiment,” “this and other embodiments,” “certain embodiments,” or the like are not necessarily mutually exclusive and are intended to indicate that a particular feature, structure, or characteristic described may be included in at least one embodiment. The appearances of such terms herein are not necessarily all referring to the same embodiment.

### BRIEF DESCRIPTION OF DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1 is a schematic representation of one example of a kidney pressure management system in accordance with one or more aspects of the invention;

FIG. 2 is a schematic representation of another example of a kidney pressure management system in accordance with one or more aspects of the invention; and

FIG. 3 is a schematic representation of another example of a kidney pressure management system in accordance with one or more aspects of the invention.

### DETAILED DESCRIPTION

Aspects of the disclosure are directed to showing a correlation between the pressure inside the kidney (exit of ureteroscope), the pressure at the entrance of the ureteroscope, and the fluid flow rate through the ureteroscope and kidney. Methods and systems of controlling the pressure inside the kidney from outside of the patient are disclosed. In accordance with certain embodiments, a two-channel ureteroscope configured with an irrigation channel unencumbered with any tools can be utilized in this method.

According to the Hagan-Poiseuille equation, the flow rate between two points is proportional to the pressure differential between these points and the cross-section of the channel connecting these points, and the flow rate is reversely proportional to the length of the channel and the viscosity of the fluid.

$$Q = δ P π {r}^{4} / 8 μ L \qquad ( 1 )$$

Q=volumetric flow rate
δP=pressure differential, i.e., δP=P(input)−P(output)
μ=dynamic viscosity of the fluid
L=length of the flow (channel length), or the distance between two points of interest
R=radius of the channel, which conducts the flow

According to this equation, in order for flow to increase, the pressure differential (δP) must increase, the inner diameter (ID) (or radius (r)) of the channel must increase, or the channel length (L) must decrease. However, both the channel ID and the channel length are (typically) constants for a given ureteroscope. The dynamic viscosity of the fluid is also a constant for ureteroscopic procedures, for instance, 0.9% saline solution (sterile) is typically used.

For a given ureteroscope with nothing inside its working channel, all components but one (δP) of equation (1) are constant, since they are fixed parameters of the ureteroscope or the fluid. These components can be combined into one parameter, which is also a constant. For purposes of illustration, this constant can be referred to as the Hydro-mechanical constant of a ureteroscope and assigned a letter U:

$$π {r}^{4} / 8 μ L = U \qquad ( 2 )$$

U can therefore be calculated. As a non-limiting example, a typical ureteroscope having a working channel ID=1.2 mm and a length of 0.7 m:

$$U = 8 * 1.02 * {10}^{- 3} * \text{.7} / π * {( 6 * {10}^{- 4} )}^{4} = 1.4 * 10 {\hat{}}^{- 10} Pa * sec / {m}^{3} = \text{.84} cm {H}_{2} O * min / mL$$

This means that for a typical ureteroscope it takes 0.84 centimeters of water column pressure to generate a flow rate of 1 mL/Min. In practice, this value may be skewed by one or more factors, including the material used for the working channel, as well as the geometry. So, for any specific ureteroscope this value may need to be defined empirically.

Then equation (1) can be expressed as:

$$Q = δ P * U \qquad ( 3 )$$

This implies that for a given ureteroscope the flow rate through the working channel is a function of the pressure drop between the input and output. Put another way, the pressure drop between the input and output of the scope is a function of the flow rate through the empty working channel of that particular scope, which can be expressed as:

$$δ P = Q / U \qquad ( 4 )$$

The pressure difference between the input to the ureteroscope having a Hydro-mechanical constant U and the pressure inside the kidney is a function of the flow rate.

$$δ P = P ( input ) - P ( kidney ) = Q / U \qquad ( 5 )$$

Then from (5):

$$P ( kidney ) = P ( input ) - Q / U \qquad ( 6 )$$

The input pressure to the ureteroscope can be measured and monitored, as well as the fluid flow rate through the ureteroscope. Therefore, equation (6) can be implemented as an algorithm for monitoring and controlling the pressure inside the kidney. For instance, the pressure before (upstream from) the scope and the fluid flow rate through the scope can be monitored and controlled.

One constraint to this approach is that the ID of the irrigation flow channel, or equivalent cross-section of the channel, must always be the same, i.e., never change, during the procedure. Any device, such as a laser fiber, guidewire, or basket would change this cross-section and make equation (6) invalid. The equivalent cross-section (to an ID) implies that the channel does not have to be of any specific shape, but it must be open to fluid flow and not change during the procedure. The irrigation channel must therefore be empty, i.e., free from internal structures or obstructions. This condition cannot be met by conventional ureteroscopes since the working channel is used for delivery of various devices. According to at least one embodiment a ureteroscope configured with at least two channels is provided, and in another embodiment a ureteroscope configured with two channels is provided. One channel is configured for irrigation only, and at least one other channel can be used for tools, non-limiting examples of which include a laser fiber, a basket, and/or a guidewire. The second channel may potentially also be used for either additional natural drainage, or for a forced aspiration with negative pressure by, for example, a manual syringe or a pump.

If forced aspiration is used, a minimal pressure inside the kidney also has to be controlled so that the pressure inside the kidney does not drop below its natural level. According to equation (6) the pressure inside the kidney can be constantly controlled by controlling the input pressure and the flow rate. Even if the input pressure is higher, as long as the flow rate is high enough, there is a significant pressure drop on the ureteroscope itself to keep the pressure inside the kidney wider the safety threshold. If a clog develops in the drainage from the kidney, the pressure inside the kidney starts to increase. This (per equation (6) will cause the input pressure to increase and the flow rate to decrease. A control mechanism according to aspects of the invention would detect this change and control one or more components of the system to thus bring the kidney pressure hack to a target value. In accordance with various embodiments, a control mechanism is provided that utilizes equation (6).

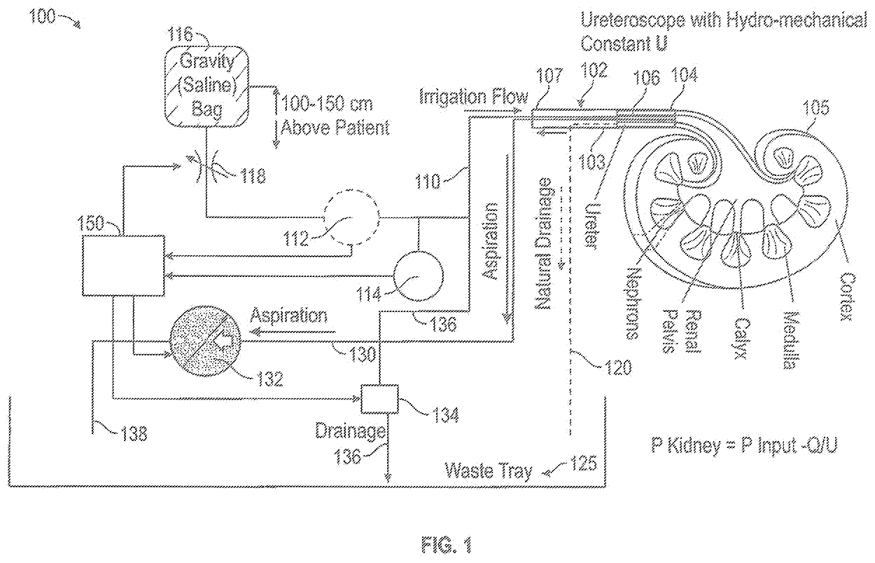

FIG. 1 is one example of a kidney pressure management system 100 in accordance with at least one embodiment, and includes a diagram outlining one example of an approach to fluid management, in this example configuration, the high fluid flow rate system is implemented with a conventional suspended irrigation bag. As explained in further detail below, a fluid flow control valve is used in combination with the irrigation bag of system 100.

System 100 comprises an irrigation channel 110 configured with a pressure sensor 114, an aspiration channel 130 in fluid communication with a drain reservoir 125 (also referred to as a waste tray), and a controller ISO. A ureteroscope 102 of system 100 includes at least a portion of the irrigation channel 110 and the aspiration channel 130 and is configured to be inserted into a ureter 106 of a kidney 105. Under a method of the present disclosure, the ureteroscope 102 may be inserted into a patient's body.

The irrigation channel 110 has a proximal end and a distal end, where the distal end is in fluid communication with an interior of the kidney 105 so as to deliver irrigation fluid to the interior of the kidney 105. The irrigation channel 110 is configured to be free from internal structures or obstructions, including tools such as a laser fiber, a guidewire, and a stone retrieval basket. It is to be appreciated that this list is not exhaustive and that irrigation channel 110 is also free from other tools that am also within the scope of this disclosure, including graspers, snares, forces, flexible needles, and balloon tools. Having the irrigation channel 110 be free from internal structures or obstructions (during a procedure) is necessary to establish the conditions for using equation (6) above to effectively control the pressure within the kidney.

The irrigation channel 110 is configured with a pressure sensor 114. Measuring pressure along the irrigation channel 110 with sensor 114 is much simpler and less expensive than having a pressure sensor inserted into the kidney directly, or having the pressure sensor positioned at the tip of the scope, where it is also susceptible to damage. The pressure sensor 114 is configured to sense or otherwise measure a pressure within the irrigation channel 110 and is in communication with the controller 150 (and the controller 150 is in communication with the pressure sensor 114). Pressure sensor 114 in some embodiments is positioned along the irrigation channel 110 and is in direct contact (e.g., via a port) with the irrigation fluid in the irrigation channel 110. The pressure sensor 114 is positioned in between the irrigation bag 116 (i.e., source of irrigation fluid) or irrigation flow control valve 118 and the terminal (distal) end of the irrigation channel 110. According to some embodiments, a flexible sheath 103 of the ureteroscope 102 has a central passageway for receiving at least a portion of the aspiration and irrigation channels. The flexible sheath 103 has a proximal end 107 and a distal end 104, the distal end 104 being in fluid communication with the interior of the kidney (when fully in position for a procedure). According to some embodiments, the pressure sensor 114 is positioned upstream from the proximal end 107 of the flexible sheath 103. In some embodiments, the pressure sensor 114 is positioned anywhere from adjacent to the proximal end 107 of the flexible sheath 103 to a distance of up to 2 meters upstream from the proximal end 107.

As previously mentioned, system 100 is provided with a source of irrigation fluid configured as a conventional suspended irrigation bag 116 (also referred to as a gravity bag or fluid bag). In some embodiments, an irrigation fluid flow control valve 118 is in fluid communication with this source of irrigation fluid and is operable to control the flow rate of irrigation fluid in the irrigation channel 110. To this end, the irrigation fluid flow control valve 118 is in communication with the controller 150 and is configured with or otherwise equipped with variable flow rate capabilities.

In some embodiments, the irrigation channel 110 is also optionally configured with a flow rate sensor, as indicated by the dotted line structure of flow rate sensor 112 in FIG. 1. When implemented, the flow rate sensor 112 is configured to sense or otherwise measure an irrigation fluid flow rate within the irrigation channel 110 and is in communication with the controller 150. The flow rate sensor 112 is positioned in between the source of irrigation fluid (e.g., irrigation bag 116) or irrigation flow control valve 118 and the terminal (distal) end of the irrigation channel 110. The optional flow rate measurements may be used by controller 150 in equation (6) when calculating a kidney pressure, as described in further detail below. Flow rate measurements may be used, for instance, when an irrigation pump has not been calibrated or in other instances where the fluid flow rate in the irrigation channel is unknown or changing.

The aspiration channel 130 of system 100 has a proximal end and a distal end, where the distal end is in fluid communication with the interior of the kidney 105 so as to remove irrigation fluid from the interior of the kidney 105. The drainage channel 138 is configured to provide fluid communication between the aspiration channel 130 and the drain reservoir 125. The aspiration pump 132 is in fluid communication with the aspiration channel 130 and is configured to pump irrigation fluid from the distal end toward the proximal end of the aspiration channel 130 to drainage channel 138 and into the drain reservoir 125. In accordance with at least one embodiment, the aspiration pump 132 is configured as a variable speed pump. As indicated in FIG. 1, the proximal end of the aspiration channel 130 is in fluid communication with the drain reservoir 125 and the aspiration pump 132 is positioned between the proximal end of the aspiration channel 130 and the distal end of the aspiration channel 130.

System 100 also comprises a drainage channel 136 configured with a safety pressure relief valve 134 (also referred to as simply a “safety valve”). The drainage channel 136 is configured to provide fluid communication between the irrigation channel 110 and the drain reservoir 125. The safety valve 134 is operable to allow or stop (i.e., control) a flow of irrigation fluid from the irrigation channel 110 to the drain reservoir 125. During regular operation safety valve 134 is in a closed position so that irrigation fluid flows through the irrigation channel 110 into the kidney. The drainage channel 136 may be used as a safety measure in instances where the calculated kidney pressure is exceedingly high and irrigation fluid in the irrigation channel needs to be immediately directed to the drainage channel 136. The controller 150 will then send a signal to open safety valve 134, thus providing fluid communication between the irrigation channel 110 and the drain reservoir 125.

System drainage is also supplied by a drainage flow path 120 (also referred to as natural drainage) that exists between the flexible sheath 103 and the ureter 106. Drainage into the drain reservoir 125 is also provided through an aspiration pump 132 via drainage channel 138. Drainage channel 136, drainage flow path 120, and drainage channel 138 are all fluidly connected to the drain reservoir 125.

Controller 150 is configured to determine a fluid flow rate of irrigation fluid in the irrigation channel 110. For example, the controller 150 may receive a value for the fluid flow rate of the irrigation fluid in the irrigation channel 110 as an input from an operator or the irrigation fluid flow control valve 118 or other device (e.g., irrigation pump) configured to send the irrigation fluid flow rate value to the controller 150. As noted previously, in some embodiments the pressure drop across the irrigation channel 110 can be considered constant and therefore the fluid flow rate within the irrigation channel 110 can be considered a constant. For example, in instances where a variable speed pump is used for pumping irrigation fluid through the irrigation channel (e.g., system 200 described below), a well-calibrated irrigation pump would imply that a fluid flow rate sensor (and fluid flow rate measurements) can be omitted. A flow rate could be calculated or otherwise obtained based on flow rate data from the irrigation pump.

Controller 150 is also configured to receive a pressure measurement value from the pressure sensor 114 of the irrigation channel 110. The controller 150 calculates a pressure within the kidney based at least in part on the determined fluid flow rate of the irrigation fluid in the irrigation channel 110 and the pressure measurement of the fluid in the irrigation channel 110 (e.g., using equation (6) above).

The Hydro-mechanical constant U of equation (6) may also be calculated (or directly input by an operator) by the controller 150 based on the physical parameters of the ureteroscope (i.e., the channel ID and length), which may also be input to the controller 150 by an operator. The controller 150 then compares the calculated kidney pressure to a target kidney pressure (e.g., 10-40 mm Hg) and, based on the comparison, controls at least one of a fluid flow rate of irrigation fluid in the irrigation channel 110 and a fluid flow rate of irrigation fluid in the aspiration channel 130, as explained in more detail below. For example, controller 150 is also in communication with the irrigation fluid flow control valve 118 and the aspiration pump 132 and can send control commands to either or both of these devices to control the flow rate of irrigation fluid in the respective irrigation channel 110 and aspiration channel 130.

According to one embodiment, the Hydro-mechanical constant U of equation (6) can be calculated or otherwise obtained by performing a calibration procedure prior to a ureteroscopy procedure. During this calibration procedure irrigation flow of a known rate may be sent through the ureteroscope and directly into drainage. The pressure output is then zero, and the input pressure is equal to the actual pressure drop on the scope, which makes U of equation (6) easy to calculate.

The controller 150 (also referred to as a control system) may comprise one or more digital or analog processors (CPU) with memory (or memories), circuitry, a user interface, and/or other physical components including hard-wired and/or programmable devices as will be appreciated by those skilled in the art. In the present description and in the claims, it is indicated that the controller is “configured” or “programmed” to execute certain steps. This may be achieved in practice by any means which allow configuring or programming the controller. For instance, in instances where the controller comprises one or more CPUs, one or more programs (e.g., software are stored in an appropriate memory. The program or programs contain instructions which, when executed by the controller, cause the controller to execute the steps described and/or claimed in connection with the controller.

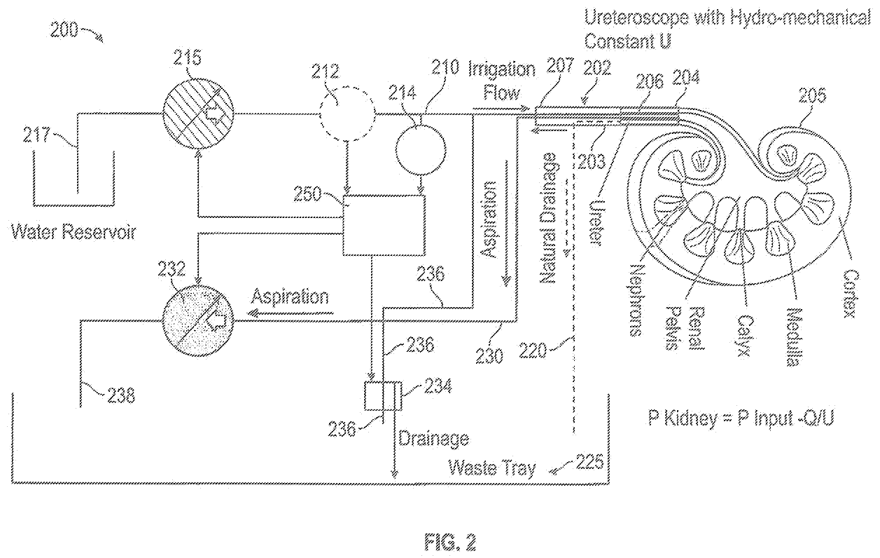

To engage system 100 in a ureteroscopy procedure once the ureteroscope is introduced into a ureter 106 of a subject, an operator such as a doctor first initiates a flow of irrigation fluid through the irrigation channel 110, which can be done using controller 150 or manually by the operator. This is accomplished by actuating or otherwise controlling a flow control valve or pump (e.g., control valve 118, or irrigation pump 215 of system 200 discussed below in reference to FIG. 2). The flow control valve or pump can be set to initiate a predetermined or target flow rate for the irrigation fluid in the irrigation channel 110. In some embodiments, this is in a range of 20-100 mL/min inclusive. In certain embodiments, the fluid flow rate of irrigation fluid is in a range of 80-100 ML/min inclusive. In other embodiments, the fluid flow rate of irrigation fluid is 10 mL/min and in some embodiments, the fluid flow rate of irrigation fluid is 20 mL/min. Irrigation fluid then flows from the proximal end to the distal end of the irrigation channel 110 and into the interior of the kidney 105.

Irrigation fluid can be removed from the kidney 105 several ways. As previously mentioned, the ureteroscope 102 includes at least a portion of the irrigation channel 110 and the aspiration channel 130 and is configured to be inserted into a ureter 106 of a kidney 105. According to some embodiments, the flexible sheath 103 of the ureteroscope 102 has a central passageway for receiving at least a portion of the aspiration and irrigation channels, and the flexible sheath is configured such that irrigation fluid can be drained into the drain reservoir 125 via a drain flow path 120 (also labeled as natural drainage in FIG. 1) between the flexible sheath 103 and the ureter 106. This is therefore at least one mechanism for irrigation fluid to egress the kidney 105. Irrigation fluid can flow into the drain reservoir 125 through drain flow path 120 through fluid pressure created by incoming irrigation fluid into the kidney 105 without the use of a pump (e.g., without the use of aspiration pump 132). As the procedure commences and the kidney fills with more fluid, the controller ISO can activate aspiration pump 132, which pumps irrigation fluid from the distal end toward the proximal end of the aspiration channel 130 to drainage channel 138 and into the drain reservoir 125.

Once the flow of irrigation fluid has commenced from the irrigation source, the controller 150 receives pressure measurements from pressure sensor 114. The pressure inside the kidney 105 is calculated by controller 150 based at least in part on the pressure measurement and the determined fluid flow rate of the irrigation fluid, as previously discussed. Controller 150 compares the calculated kidney pressure to a target kidney pressure value and then based on this comparison, the controller 150 controls at least one of a flow rate of irrigation fluid in the irrigation channel 110 and a flow rate of irrigation fluid in the aspiration channel 130.

According to one embodiment, the target kidney pressure value is in a range of 10-40 mm Hg inclusive. In some embodiments, if the calculated kidney pressure is greater than the target kidney pressure, then the controller 150 is configured to increase the fluid flow rate of irrigation fluid in the aspiration channel 130, decrease the fluid flow rate of irrigation fluid in the irrigation channel 110, or both (i.e., increase the fluid flow rate of irrigation fluid in the aspiration channel 130 and decrease the fluid flow rate of irrigation fluid in the irrigation channel 110). For example, the controller 150 is configured to control the aspiration pump 132 so as to increase the fluid flow rate of irrigation fluid in the aspiration channel 130 and control the irrigation fluid flow control valve 118 so as to decrease the fluid flow rate of irrigation fluid in the irrigation channel 110. As mentioned previously, the aspiration pump 132 is configured as a variable speed pump and the controller 150 controls the aspiration pump 132 by powering on or increasing a speed of the variable speed so as to increase the fluid flow rate of irrigation fluid in the aspiration channel 130. As previously mentioned, irrigation fluid flow control valve 118 is also configured with variable flow rate capabilities. The controller 150 may therefore control the irrigation fluid flow control valve 118 by restricting or closing the valve to decrease the rate at which irrigation fluid enters the kidney 105 from the irrigation channel 110.

Increasing the fluid flow rate in the aspiration channel 130 creates a negative pressure in the aspiration channel 130, with irrigation fluid now being directed by the aspiration pump 132 through drainage channel 138 to the drain reservoir 125. The negative pressure reduces the pressure inside the kidney 105. Reducing the fluid flow rate of irrigation fluid in the irrigation channel 110 reduces the amount of fluid entering the kidney, which can allow greater drainage to occur, through drainage flow path 120 and/or through drainage channel 138, thereby reducing the pressure within the kidney 105.

In some embodiments, controller 150 may control the irrigation fluid flow control valve 118 and/or the safety valve 134 so as to decrease the fluid flow of irrigation fluid in the irrigation channel 110 when the aspiration channel 130 is clogged. A clogged aspiration channel create an increasing pressure in the kidney 105 (and causes the calculated kidney pressure value to be greater than the target kidney pressure value) despite the controller 150 having sent a control signal to increase the speed or operation of the aspiration pump 132. As mentioned previously, safety valve 134 is operable to control a flow of irrigation fluid from the irrigation channel 110 to the drain reservoir 125 and is normally closed so that irrigation fluid is directed through the irrigation channel 110 into the kidney 105 but can function as a safety measure in instances where the calculated pressure in the kidney is greater than the target kidney pressure, such as in instances where the calculated pressure in the kidney is much greater than the target kidney pressure and actually exceeds a (predetermined) maximum kidney pressure. For example, if the target kidney pressure is in a range of 10-40 mm Hg, then the maximum kidney pressure may be 45 mm Hg (and above). In such instances where the calculated pressure exceeds this maximum kidney pressure, the controller 150 is configured to control safety valve 134 by opening the safety valve 134 so as to allow fluid communication between the irrigation channel 110 and the drain reservoir 125.

Pressure sensor 114 and optional flow rate sensor 112 can be configured or controlled by the controller 150 to take measurements, including continuous or periodic measurements once the procedure has commenced. This measurement data is received by the controller 150 and is used to calculate the interior pressure of the kidney 105. As previously mentioned, according to some embodiments, the controller 150 receives a fluid flow rate measurement value from a flow rate sensor 112 of the irrigation channel 110.

In accordance with at least one embodiment, if the calculated kidney pressure is less than or lower than the target kidney pressure, then the controller 150 is configured to decrease the fluid flow rate of irrigation fluid in the aspiration channel 130, increase the fluid flow rate of irrigation fluid in the irrigation channel 110, or both (i.e., decrease the fluid flow rate of irrigation fluid in the aspiration channel 130 and increase the fluid flow rate of irrigation fluid in the irrigation channel 110). For example, controller 150 is configured to control the aspiration pump 132 so as to decrease the fluid flow rate of irrigation fluid in the aspiration channel 130 and control the irrigation fluid flow control valve 118 so as to increase the fluid flow rate of irrigation fluid in the irrigation channel 110. As mentioned previously, the aspiration pump 132 is configured as a variable speed pump and the controller 150 controls the aspiration pump 132 by powering off or decreasing a speed of the variable speed so as to decrease the fluid flow rate of irrigation fluid in the aspiration channel 130. Controller 150 is configured to control the irrigation fluid flow control valve 118 by opening (i.e., opening further) the valve 118 (or by increasing the speed of the variable speed pump 215 as described below in reference to system 200) so as to increase the rate at which irrigation fluid enters the kidney 105 from the irrigation channel 110. One or both of these actions by the controller 150 allows irrigation fluid to accumulate in the kidney 105 and thereby increase the internal pressure inside the kidney 105.

FIG. 2 is another example of a kidney pressure management system 200 in accordance with another embodiment, and is similar to system 100 of FIG. 1, but in this example configuration, the high fluid flow rate system is implemented with an irrigation pump 215 that is fluidly connected to the irrigation channel 210 (and a source of irrigation fluid, e.g., irrigation source 217) instead of the irrigation fluid flow control valve 118 used in combination with the irrigation bag 116 (irrigation source) of system 100 in FIG. 1. The irrigation pump 215 is in fluid communication with a source of irrigation fluid 217 (water reservoir). The irrigation pump 215 is also in communication with the controller 250, and is operable to control the flow rate of irrigation fluid in the irrigation channel 210. The irrigation pump 215 is configured as a variable speed pump.

System 200 operates in a similar manner as system 100 of FIG. 1 and in the interest of brevity, will not be repeated here. The main difference is that instead of controlling the irrigation fluid flow control valve 118, the controller 250 controls the irrigation pump 215 by adjusting the speed of the pump to decrease or increase the rate at which irrigation fluid enters the kidney 205 from the irrigation channel 210. For example, when the calculated kidney pressure is greater than the target kidney pressure value then the controller 250 may decrease the fluid flow rate of irrigation fluid in the irrigation channel 210 by powering off or decreasing a speed of the irrigation pump 215. When the calculated kidney pressure is less than the target kidney pressure value then the controller 250 may increase the fluid flow rate of irrigation fluid in the irrigation channel 210 by powering on or increasing a speed of the irrigation pump 215. Another difference system 200 has from system 100 is regarding the maximum input pressure. In the configuration of system 100 with the suspended bag, the maximum pressure is limited by the height, of the bag. The flow rate would then fluctuate as a function of the kidney pressure (Pᵢₙₚᵤₜ—constant; if P_{kidney} increases, the flow rate decreases per equation (6)). Since the flow rate fluctuates, in order to control the pressure in the kidney, the flow rate may be monitored by a flow meter (e.g., flow meter 112) and utilized by equation (6).

Some pumps, however, can generate a substantially high pressure so that they can maintain the constant flow rate in a full range of input pressures required for a procedure. In this case, if the pump's variable speed is graduated, for example, in mli/min flow and the flow rate is calibrated precisely, it is possible to use the pump flow rate setting instead of the direct flow rate measurement. One benefit of this configuration is that the flow meter itself is not necessary. In one specific experiment performed by Applicant, an irrigation pump (model PP-606 Precision Peristaltic Pump, commercially available from Bianca Pumps, Temecula, California. USA) was used, which is capable of producing 0-111 mL/min of a variable fluid flow rate at a high pressure. The irrigation fluid flow rate was set to 80 mL/min and the pressure drop on the scope at this rate was within a range of 250-400 cm H₂O. When the kidney pressure increased, the pump was able to generate enough input pressure to maintain the irrigation fluid flow rate such that direct monitoring of the irrigation fluid flow rate was not necessary, and instead, the pump flow rate setting was used as input by the controller.

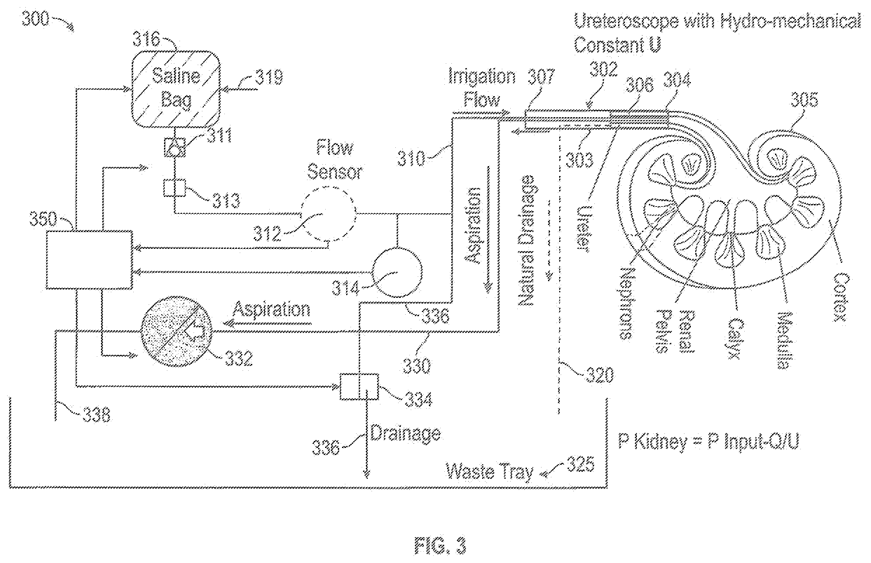

FIG. 3 is another example of a kidney pressure management system 300 in accordance with another embodiment, and is similar to system 100 of FIG. 1 and system 200 of FIG. 2, but in this example configuration, the high fluid flow rate system is implemented with a variable compression device or mechanism 319, a stop valve 313, and a check valve 311 positioned on the irrigation channel 310. One or more of these devices can be used in combination with the irrigation bag 316 (irrigation source) to control the flow of irrigation fluid in the irrigation channel 310.

The variable compression device 319 is configured to apply pressure or otherwise apply a compressive force on the irrigation bag 316 to control the flow of irrigation fluid in the irrigation channel 310. For instance, increasing the compressive force on the irrigation bag 316 results in an increase in the flow rate of irrigation fluid in the irrigation channel 310, and decreasing the compressive force has the opposite effect, i.e., decreases the fluid flow rate of irrigation fluid in the irrigation channel 310. The stop valve 313 can also assist with this functionality by allowing (when open) fluid communication between the irrigation source 316 and the irrigation channel 310 or discontinuing (when closed) fluid communication between the irrigation source 316 and the irrigation channel 310. The check valve 311 allows for the irrigation bag 316 to be placed at any level with respect to the patient (i.e., not just 40 cm above the patient) and prevents irrigation fluid from the kidney 305 from flowing backward into the irrigation bag 316. As indicated in FIG. 3, each of the variable compression device 319, stop valve 313, and check valve 311 is in communication with the controller 350 and is operable (alone or in combination) to control a flow rate of irrigation fluid in the irrigation channel 310.

System 300 operates in a similar manner as systems 100 and 200 of FIGS. 1 and 2, and in the interest of brevity, will not be repeated here. The main difference is that instead of controlling the irrigation fluid flow control valve 118 or the irrigation pump 215, the controller 350 controls the variable compression device 319 to decrease or increase the rate at which irrigation fluid enters the kidney 305 from the irrigation channel 310. The controller 350 may also control the stop valve 313 and/or check valve 311 to assist with this functionality. For example, for a particular utereoscopy, a doctor or the controller 350 may initiate the procedure by first opening the stop valve 313 in combination with establishing a desired flow rate of irrigation fluid by applying the compressive force via the variable compression device 319.

Although not expressly shown in the figures, in other embodiments systems 100, 200, and 300 may also include an unclogging feature, i.e., the systems may be configured to redirect irrigation fluid into the aspiration channel. For instance, a valve of drainage channel 136, 236, and 336 can be configured to direct irrigation fluid from the irrigation channel to the aspiration channel. In some instances, this same valve may also be configured to direct irrigation fluid to the drain reservoir 125, 225, 325 from the irrigation channel.

### EXAMPLES

Functions and advantages of the embodiments of the systems and techniques disclosed herein may be more fully understood based on the examples described below. The following examples are intended to illustrate various aspects of the disclosed kidney pressure management system, but are not intended to fully exemplify the full scope thereof.

### Example 1—System 100 of FIG. 1 Operates as Follows

A doctor opens a flow control valve (e.g., valve 118) to the level of desirable flow rate;
The irrigation fluid runs through the irrigation channel 110 of the two-channeled scope 102 into kidney 105 and from the kidney 105 runs out into waste through the ureter/urethra around the scope body (Natural Drainage, drainage flow path 120);
If the calculated pressure inside the kidney 105 rises above the target pressure value (as determined by controller 150), the controller
   turns on or increases the speed of the aspiration pump 132, and/or
   restricts or closes the variable flow control valve 118 on irrigation line 110;
If the calculated pressure inside the kidney 105 is less than the target pressure value, the controller
   shuts down or slows down the speed of the aspiration pump 132, and/or
   opens or further opens the variable flow control valve 118 on irrigation line 110

### Example 2—System 200 of FIG. 2 Operates as Follows

A doctor turns the irrigation pump 215 and sets it to a predetermined value;
The irrigation fluid runs through the irrigation channel 210 of the two-channeled scope 202 into kidney 205 and from the kidney 205 runs out into waste through the ureter/urethra around the scope body (Natural Drainage, drainage flow path 220);
If the calculated pressure inside the kidney 205 rises above the target pressure value (as determined by controller 250), the controller 250
   turns on or increases the speed of the aspiration pump 232, and/or
   slows down or shuts down irrigation pump 215
If the calculated pressure inside the kidney 205 is less than the target pressure value, the controller
   turns off or decreases the speed of the aspiration pump 232, and/or
   speeds up or shuts off the irrigation pump 215

The aspects disclosed herein in accordance with the present invention, are not limited in their application to the details of construction and the arrangement of components set forth in the following description or illustrated in the accompanying drawings. These aspects are capable of assuming other embodiments and of being practiced or of being carried out in various ways. Examples of specific implementations are provided herein for illustrative purposes only and are not intended to be limiting. In particular, acts, components, elements, and features discussed in connection with any one or more embodiments are not intended to be excluded from a similar role in any other embodiments.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. In addition, in the event of inconsistent usages of terms between this document and documents incorporated herein by reference, the term usage in the incorporated reference is supplementary to that of this document; for irreconcilable inconsistencies, the term usage in this document controls. Moreover, titles or subtitles may be used in the specification for the convenience of a reader, which shall have no influence on the scope of the present invention.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description and drawings are by way of example only.

What is claimed is:

1. A system for controlling pressure within a kidney, comprising:
an irrigation channel having a proximal end and a distal end and configured with a pressure sensor that is configured to measure pressure within the irrigation channel, the distal end of the irrigation channel in fluid communication with an interior of a kidney so as to deliver irrigation fluid to the interior of the kidney;
an aspiration channel having a proximal end and a distal end, the aspiration channel in fluid communication with a drain reservoir, the distal end of the aspiration channel in fluid communication with the interior of the kidney so as to remove irrigation fluid from the interior of the kidney; and
a controller in communication with the pressure sensor and configured to:
determine a fluid flow rate of irrigation fluid within the irrigation channel,
receive a pressure measurement value from the pressure sensor,
calculate a pressure within the interior of the kidney based at least in part on the determined fluid flow rate and the pressure measurement,
compare the calculated kidney pressure to a target kidney pressure value, and
based on the comparison, control at least one of the fluid flow rate of irrigation fluid in the irrigation channel and a fluid flow rate of irrigation fluid in the aspiration channel.

2. The system of claim 1, wherein the irrigation channel is free from internal structures or obstructions.

3. The system of claim 2, wherein the irrigation channel is free from a laser fiber, a guidewire, and a stone retrieval basket.

4. The system of claim 1, further comprising a flexible sheath having a central passageway for receiving at least a portion of the aspiration and irrigation channels and is configured to be inserted into a ureter, the flexible sheath configured such that irrigation fluid can be drained into the drain reservoir via a drainage flow path between the flexible sheath and the ureter.

5. The system of claim 4, wherein the flexible sheath has a proximal end and a distal end, the distal end of the flexible sheath in fluid communication with the interior of the kidney and the pressure sensor is positioned upstream from the proximal end of the flexible sheath.

6. The system of claim 1, wherein when the calculated kidney pressure is greater than the target kidney pressure value the controller is configured to
increase the fluid flow rate of irrigation fluid in the aspiration channel,
decrease the fluid flow rate of irrigation fluid in the irrigation channel, or
increase the fluid flow rate of irrigation fluid in the aspiration channel and decrease the fluid flow rate of irrigation fluid in the irrigation channel.

7. The system of claim 6, further comprising:
an aspiration pump in fluid communication with the aspiration channel and configured to pump irrigation fluid from the distal end toward the proximal end of the aspiration channel, and the controller is configured to control the aspiration pump so as to increase the fluid flow rate of irrigation fluid in the aspiration channel, and
one of an irrigation pump or an irrigation fluid flow control valve, each of which is:
in fluid communication with a source of the irrigation fluid,
in communication with the controller, and
is operable to control the fluid flow rate of irrigation fluid in the irrigation channel, and
the controller is configured to control at least one of the irrigation pump and the irrigation fluid flow control valve so as to decrease the fluid flow rate of irrigation fluid in the irrigation channel.

8. The system of claim 7, wherein
the aspiration pump is configured as a variable speed pump and the controller controls the aspiration pump by powering on or increasing a speed of the variable speed,
the irrigation pump is configured as a variable speed pump and the controller controls the irrigation pump by powering off or decreasing a speed of the variable speed, and
the controller controls the irrigation fluid flow control valve by restricting or closing the irrigation fluid flow control valve.

9. The system of claim 6, further comprising a drainage channel configured to provide fluid communication between the irrigation channel and the drain reservoir, the drainage channel configured with a safety valve operable to control a flow of irrigation fluid from the irrigation channel to the drain reservoir.

10. The system of claim 9, wherein the controller is configured to control the safety valve by opening the safety valve so as to allow fluid communication between the irrigation channel and the drain reservoir.

11. The system of claim 1, wherein when the calculated kidney pressure is less than the target kidney pressure value, the controller is configured to
decrease the fluid flow rate of irrigation fluid in the aspiration channel,
increase the fluid flow rate of irrigation fluid in the irrigation channel, or
decrease the fluid flow rate of irrigation fluid in the aspiration channel and increase the fluid flow rate of irrigation fluid in the irrigation channel.

12. The system of claim 1, further comprising a fluid flow rate sensor configured to measure an irrigation fluid flow rate within the irrigation channel, and the controller is further configured to receive a fluid flow rate measurement value from the fluid flow rate sensor and calculate the pressure within the interior of the kidney based at least in part on the fluid flow rate measurement.

13. The system of claim 1, wherein the target kidney pressure value is in a range of 10-40 mm Hg inclusive.

14. A method for controlling pressure within a kidney, comprising:
directing irrigation fluid through an irrigation channel to an interior of the kidney;
removing irrigation fluid from the interior of the kidney and directing the irrigation fluid through an aspiration channel toward a drain reservoir;
determining a fluid flow rate of irrigation fluid within the irrigation channel;
measuring a pressure within the irrigation channel;
calculating a pressure within an interior of the kidney based at least in part on the determined fluid flow rate and the pressure measurement;
comparing the calculated kidney pressure to a target kidney pressure value; and
based on the comparison, controlling at least one of a fluid flow rate of irrigation fluid in the aspiration channel and a fluid flow rate of irrigation fluid in the irrigation channel.

15. The method of claim 14, wherein when the calculated kidney pressure is greater than the target kidney pressure value the method comprises at least one of
increasing a fluid flow rate of irrigation fluid in the aspiration channel, and
decreasing a fluid flow rate of irrigation fluid in the irrigation channel.

16. The method of claim 15, wherein
increasing the fluid flow rate of irrigation fluid in the aspiration channel includes at least one of powering on or increasing a speed of an aspiration pump in fluid communication with the aspiration channel, and
decreasing the fluid flow rate of irrigation fluid in the irrigation channel includes at least one of
powering off or decreasing a speed of an irrigation pump that is in fluid communication with a source of irrigation fluid, and
restricting or closing an irrigation fluid flow control valve that is in fluid communication with the source of irrigation fluid.

17. The method of claim 15, further comprising directing irrigation fluid through a drainage channel that is configured to provide fluid communication between the irrigation channel and the drain reservoir.

18. The method of claim 14, wherein when the calculated kidney pressure is less than the target kidney pressure value, the method comprises at least one of
decreasing a fluid flow rate of irrigation fluid in the aspiration channel, and
increasing a fluid flow rate of irrigation fluid in the irrigation channel.

19. The method of claim 18, wherein
decreasing the fluid flow rate of irrigation fluid in the aspiration channel includes powering off or decreasing a speed of an aspiration pump in fluid communication with the aspiration channel, and
increasing the fluid flow rate of irrigation fluid in the irrigation channel includes at least one of
powering on or increasing a speed of an irrigation pump that is in fluid communication with a source of irrigation fluid, and
opening an irrigation fluid flow control valve that is in fluid communication with the source of irrigation fluid.

20. The method of claim 14, further comprising measuring the fluid flow rate of irrigation fluid within the irrigation channel and calculating the pressure within the interior of the kidney based at least in pan on the fluid flow rate measurement.

21. The method of claim 14, further comprising
providing a flexible sheath having a central passageway for receiving at least a portion of the aspiration and irrigation channels, and
positioning the flexible sheath within an ureter such that irrigation fluid can be drained into the drain reservoir frim a drainage flow path between the flexible sheath and the ureter.

22. The method of claim 21, wherein
measuring the pressure within the irrigation channel includes measuring with a pressure sensor in the irrigation channel, and
the flexible sheath has a proximal end and a distal end, the distal end of the flexible sheath in fluid communication with the interior of the kidney, and
the method further comprises
providing the irrigation channel, the irrigation channel configured such that the pressure sensor is positioned upstream from the proximal end of the flexible sheath.

23. The method of claim 14, wherein the target kidney pressure value is in a range of 10-40 mm Hg inclusive.

24. The method of claim 14, further comprising providing a ureteroscope that includes the irrigation channel and the aspiration channel, wherein the irrigation channel is configured to be free from internal structures or obstructions.
